(19) United States
(12) Patent Application Publication
(10) Pub. No.: US 2025/0256105 A1
(43) Pub. Date: Aug. 14, 2025

(54) NEUROMODULATION OF TWO OR MORE NEURONAL TARGETS IN TREATMENT OF A MEDICAL CONDITION

(71) Applicant: RESHAPE LIFESCIENCES, INC., St. Paul, MN (US)

(72) Inventors: Jonathan James WAATAJA, Plymouth, MN (US); Raj NIHALANI, Irvine, CA (US)

(73) Assignee: RESHAPE LIFESCIENCES, INC., St. Paul, MN (US)

(21) Appl. No.: 18/877,731

(22) PCT Filed: Jun. 22, 2023
(86) PCT No.: PCT/US2023/068891

§ 371 Date: Dec. 20, 2024

Related U.S. Application Data
(60) Provisional application No. 63/354,489, filed on Jun. 22, 2022.

(51) Int. Cl. A61N 1/36

(52) U.S. Cl. CPC A61N 1/36167; A61N 1/36053; A61N 1/36139

(57) ABSTRACT

The present disclosure is directed to the neuromodulation of two or more neuronal targets in treatment of a medical condition. The neuronal targets can be sympathetic nerves, parasympathetic nerves or a combination of sympathetic and parasympathetic nerves. The neuromodulation of the neuronal targets can comprise stimulation of the neuronal target, a conduction block of the neuronal targets or a combination of stimulation and conduction blocks of the neuronal targets. The neuromodulation is performed by one or more neuroregulators which can comprise electrical or non-electrical neuroregulators. The neuromodulation of each of the neuronal targets can include the same or different start/end times, the same or different durations, and/or the same or different neuromodulation patterns. The neuromodulation of the first neuronal target works in cooperation with the neuromodulation of the second neuronal target to treat a medical condition.

### FIELD OF THE DISCLOSURE

The present disclosure is directed to neuromodulation and, more particularly, to neuromodulation of two or more neuronal targets in treatment of a medical condition.

### BACKGROUND

The peripheral nervous system is composed of two major branches, the somatosensory which traffics, for example, touch and pain information to the central nervous system (CNS). The other branch consists of the autonomic nervous system which carries information of the state of internal organs to the CNS and also information from the CNS to control the internal organs. The autonomic nervous system can further be broken up into two divisions, the sympathetic and parasympathetic nervous systems. These act in an antagonistic manner. The sympathetic puts the body in a “flight or fight” mode. For example activation of the sympathetic nervous system takes blood away from the stomach and guides to muscles, slows digestion and increases release of adrenalin. The parasympathetic nervous system put the body into a “rest and digest” mode. For example its activation would guide blood to the stomach and increases digestive processes. Most, if not all internal organs are innervated by both the sympathetic and parasympathetic nervous systems.

In terms of neuromodulation (stimulation or conduction block) with the intent to control the function of an internal organ, modulation of only one arm of the autonomic nervous system may not have maximal, or very little, efficacy. This is due to the fact that when one arm is modulated the other arm may compensate for the exogenous up or down regulation of the nerve innervating the organ. Accordingly, the neuromodulation of a single neuronal target has often failed to produce an effect sufficient to treat a number of medical conditions.

### SUMMARY

The present disclosure is directed to the neuromodulation (stimulation or conduction block) of two or more neuronal targets in treatment of a medical condition. The neuronal targets can be sympathetic nerves, parasympathetic nerves or a combination of sympathetic and parasympathetic nerves. The neuromodulation of the neuronal targets can comprise stimulation of the neuronal targets, a conduction block of the neuronal targets or a combination of stimulation and conduction blocks of the neuronal targets. The neuromodulation is performed by one or more neuroregulators that can comprise electrical or non-electrical neuroregulators. The neuromodulation of each of the neuronal targets can include the same or different start/end times, the same or different durations, and/or the same or different neuromodulation patterns. The neuromodulation of the first neuronal target works in cooperation with the neuromodulation of the second neuronal target to treat a medical condition.

One aspect of the present disclosure is directed to a system for regulating at least two neuronal targets. The system includes a first neuroregulator and a second neuroregulator. The first neuroregulator applies a stimulation or a conduction block to a first neuronal target. The second neuroregulator applies a stimulator or a conduction block to a second neuronal target, which is different from the first neuronal target. The stimulation or conduction block of the first neuronal target works in cooperation with the stimulation or conduction block of the second neuronal target to treat a medical condition. In certain embodiments, each of the first and second neuroregulators are electrical neuroregulators, non-electrical neuroregulators or a combination of electrical and non-electrical neuroregulators.

Another aspect of the present disclosure is directed to a method for treating a medical condition. The method includes: (a) neuromodulating, with a stimulation or a conduction block, a first neuronal target; and (b) neuromodulating, with a stimulation or a conduction block, a second neuronal target, wherein the second neuronal target is different from the first neuronal target and wherein the neuromodulation of the first neuronal target works in cooperation with the neuromodulation of the second neuronal target to treat a medical condition.

### DESCRIPTION OF THE DRAWINGS

FIG. 1 is an exemplary schematic of a neuromodulation system for neuromodulating two or more neuronal targets according to the present disclosure.

FIG. 2 is an exemplary schematic of another neuromodulation system for neuromodulating two or more neuronal targets according to the present disclosure.

FIG. 3 is an exemplary schematic of another neuromodulation system for neuromodulating two or more neuronal targets according to the present disclosure.

FIG. 4 is a time course, in relation to a Type II Diabetes Rat Model Study described herein, of various conditions in experimental group 2.

FIG. 5 is graph, in relation to the Type II Diabetes Rat Model Study described herein, illustrating percent change in blood glucose concentration in sham or celiac branch stimulation and hepatic nerve vagotomy (experimental group 1) prior to a glucose challenge.

FIG. 6 is a graph, in relation to the Type II Diabetes Rat Model Study described herein, illustrating time course of percent change of blood glucose concentration following a glucose challenge for the sham, celiac branch stimulation and hepatic nerve vagotomy (experimental group 1) and celiac branch stimulation with concurrent delivery of 5000 Hz to the hepatic nerve (experimental group 2) arms.

FIG. 7 is a bar chart, in relation to the Type II Diabetes Rat Model Study described herein, illustrating peak glucose concentration (as measured as percentage change from baseline) following a glucose challenge for each experimental condition.

FIG. 8 is a graph, in relation to the Type II Diabetes Rat Model Study described herein, of the percentage change in blood glucose versus time for the celiac branch stimulation and simultaneous delivery of 5000 Hz to the hepatic nerve (experimental group 2) arm before a glucose tolerance test.

FIG. 9 is a graph, in relation to the Type II Diabetes Rat Model Study described herein, of the percentage change in blood glucose concentration over time with stimulation of the celiac branch and concurrent delivery of 5000 Hz to the hepatic nerve (dashed line) with two glucose challenges.

FIG. 10 is a flow chart showing the therapeutic process to control blood glucose levels.

FIG. 11 is an illustrative depiction of exemplary innervation locations for modifying blood glucose levels.

FIG. 12 is a graphical illustration of blood glucose level examples over the course of a 24-hour period.

FIG. 13 is a graph, in relation to the Type II Diabetes Study described herein, of the blood glucose concentration (mg/dL) over time (min) with stimulation of the celiac branch and concurrent delivery of 5000 Hz to the hepatic nerve 5 minutes after OGTT.

FIG. 14 is an illustration of one example of lead placements and neuroregulator controls.

FIG. 15 is a table showing co-morbidities related to diabetes.

### DETAILED DESCRIPTION

The present disclosure is directed to the neuromodulation (stimulation or conduction block) of two or more neuronal targets in treatment of a medical condition. The neuronal targets generally comprise sympathetic and/or parasympathetic nerves, which typically innervate an internal organ. One or more neuroregulators provide the desired stimulation or conduction block to the neuronal targets, which work in cooperation to treat a medical condition. In certain embodiments the two or more neuronal targets are: (a) neuromodulated over a common time period; (b) neuromodulated during overlapping time periods; (c) neuromodulated in distinct non-overlapping time periods; and/or (d) neuromodulated in any combination of (a), (b), and (c). The neuromodulation of each of the neuronal targets can include the same or different start/end times, the same or different durations, and/or the same or different neuromodulation patterns.

Stimulation neuroregulators can comprise, for example, electrical stimulators or chemical agent stimulators. Conduction block neuroregulators can comprise, for example, physical section (e.g. ablation) conduction blocks, electrical conduction blocks, electrical high frequency conduction blocks, chemical conduction blocks or an optogenetic conduction blocks obtained by delivery of an inhibitory opsin or excitatory opsin that is flashed with high frequency light. Of the noted neuroregulators, electrical stimulators, electrical conduction blocks and electrical high frequency conduction blocks can be deemed electrical neuroregulators while all other noted neuroregulators can be deemed non-electrical neuroregulators.

Medical Conditions that can be treated the cooperative neuromodulation of two or more neuronal targets include, but are not limited to: (a) heart failure; (b) inflammation; (c) hypertension; (d) pancreatitis; (e) refractory asthma; (f) urinary incontinence; (g) erectile dysfunction; (h) type II diabetes; (i) obesity; (j) Parkinson's disease; (k) refractory depression; (l) refectory epilepsy; and (m) cluster headaches.

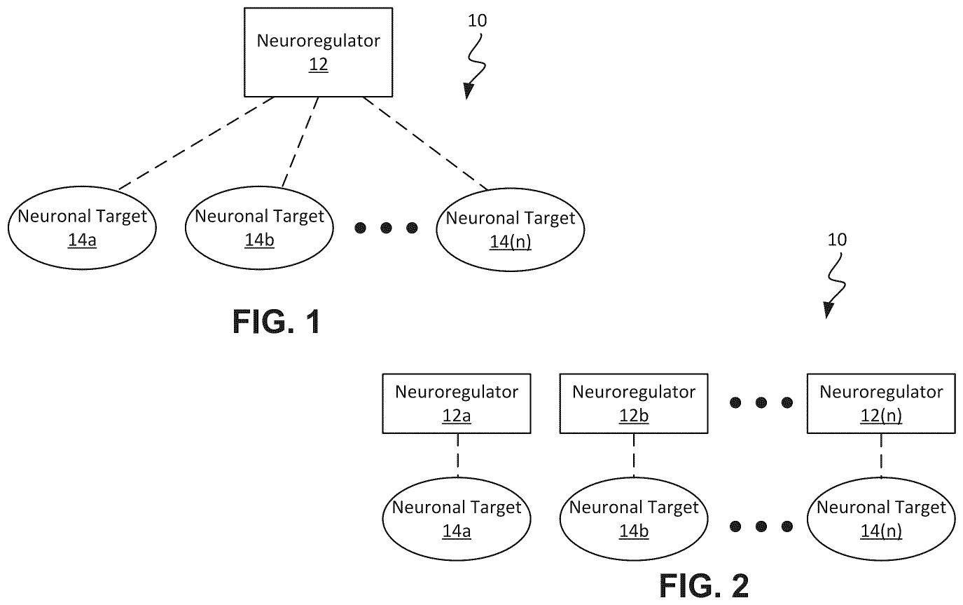

FIG. 1 illustrates a simplified schematic of a neuromodulation system 10 wherein a single neuroregulator 12 is used to neuromodulate a plurality of neuronal targets 14a, 14b . . . 14(n). FIG. 2 illustrates a simplified schematic of a neuromodulation system 10 wherein each neuronal target 14a, 14b . . . 14(n) is neuromodulated by a corresponding neuroregulator 12a, 12b . . . 12(n). Other combinations of neuroregulators and neuronal targets within a neuromodulation system are also possible. The neurodmodulation of a neuronal target operates in cooperation with the neuromodulation of the other of the two or more neuronal targets to treat a medical condition.

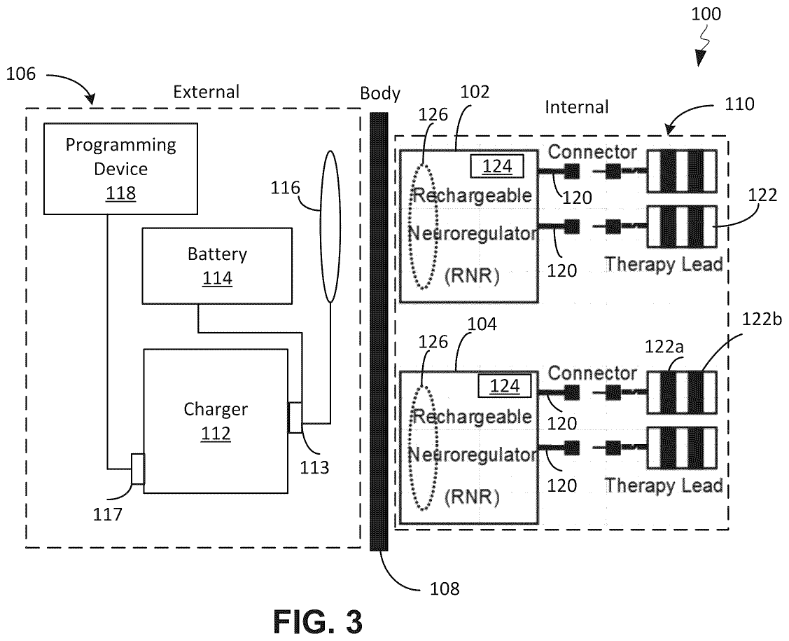

FIG. 3 provides a schematic of an exemplary neuromodulation system 100 having a first neuroregulator 102 to neuromodulate a first neuronal target and a second neuroregulator 104 to neuromodulate a second neuronal target. In this example, each of the first and second neuroregulators 102, 104 comprise electrical neuroregulators that can provide electrical stimulation and/or electrical conduction blocking. In other examples, the first neuroregulator of the neuromodulation system may comprise an electrical neuroregulator while the second neuroregulator of the neuromodulation system may comprise a non-electrical neuroregulator, e.g. the non-electrical stimulation and/or conduction block neuroregulators described in the paragraphs above.

Continuing with the example of FIG. 3, the neuromodulation system 100 generally includes an external component 106 that resides outside the body 108 and an internal component 110 that is implanted within the body 108 below the dermis. The external component 106 includes a charger 112 that is coupled, via connector 113, to a battery 114 and a transmit coil 116. The charger 112 is additionally communicatively coupled, via a communication port 117, to a programming device 118. The internal component 110 includes the first and second neuroregulators 102, 104, each of which is coupled by one or more leads 120 to one or more electrodes 122, which are placed on the neuronal targets. Each of the neuroregulators 102, 104 includes a rechargeable battery 124 and a receiving antenna 126. In certain examples, the neuroregulators 102, 104 share a common battery 124 and a common receiving antenna 126.

In the example of FIG. 3, the leads 120 comprise bipolar leads each of which is connected to first and second electrodes 122a, 122b. However, other lead and electrode configurations may be used as appropriate to a specific application. In certain examples, each of the neuroregulators 102, 104 comprises an implantable component that is independent from the other while in other examples the first and second neuroregulators 102, 104 are combined within a single implantable component. In certain examples, each of the neuroregulators 102, 104 is configured to interface with a common external component 106 while in other examples each of the neuroregulators 102, 104 interfaces with their own corresponding external component 106.

In operation, the first and second neuroregulators 102, 104 of the neuromodulation system 100 produce electrical pulses that are delivered to their respective first and second neuronal targets through the electrically conductive leads 120 and electrodes 122. In addition to delivering the electrical pulses, each of the neuroregulators 102, 104 also receives wireless command signals from the programming device 118 and can wirelessly upload data to the programming device 118 via the charger 112. Each of the neuroregulators 102, 104 is powered by their internal battery 124; in certain examples, the neuroregulators 102, 104 share a common internal battery 124. The internal battery 124 is periodically recharged by RF power that is radiated by the transmit coil 116 and picked up by the receiving antenna 126. The charger 112 provides the electrical excitation of the transmit coil 116 needed to deliver RF power to the neuroregulators 102, 104. In addition, the charger 112 serves as an interface for communications between the neuroregulators 102, 104 and the programming device 118. In certain examples, a rechargeable battery (not shown) powers the charger 112. The transmit coil 116 serves to transmit RF power transdermally from the charger 112 to the neuroregulators 102, 104. The transmit coil 116 further facilitates bi-directional RF communications between the neuroregulators 102, 104 and the charger 112. The programming device 118 enables a clinician to program each of the neuroregulators 102, 104 with a treatment schedule and with therapy parameters for delivering stimulation or conduction blocking to their respective neuronal targets.

An example of a neuromodulation system similar to the one described above, albeit with only one neuroregulator, is the MAESTRO® Rechargeable System available from ReShape Lifesciences, Inc. (St. Paul, MN). The MAESTRO® Rechargeable System is described in detail in US Patent Publication Nos.: U.S. Pat. Nos. 7,489,969; 7,167,750; 7,444,183; 7,613,515; 7,720,540; 7,630,769; 7,693,577; 7,729,771; 7,844,338; 8,046,085; 7,986,995; 8,010,204; 8,369,952; 8,538,542; 9,174,040; 8,538,533; 9,162,062, 8,862,233; 7,672,727; 8,103,349; 7,822,486; 8,140,167; 8,532,787; 8,068,918; 8,521,299; 7,917,226; 8,483,838; 8,326,426; 9,186,502; 8,483,830; 9,333,340; 6,699,275; 6,860,851; 8,825,164; US 2014/0214129; U.S. Pat. Nos. 9,393,420; 8,101,204; 8,768,469; 9,095,711; and US 2013/0237948. Each of the noted patent publications is hereby incorporated by reference in its entirety.

### Method of Use

Use of implanted pulse generator for performing the method of the invention is preferred, but treatment may conceivably be administered using external equipment on an outpatient basis, albeit only somewhat less confining than complete hospitalization. Implantation of one or more pulse generators, of course, allows the patient to be completely ambulatory, so that normal daily routine activities including on the job performance is unaffected.

The pulse generator may be programmed with programming wand and a personal computer using suitable programming software developed according to the programming needs and signal parameters which have been described herein. The intention, of course, is to permit noninvasive communication with the electronics package after the latter is implanted, for both monitoring and programming functions. Beyond the essential functions, the programming software should be structured to provide straightforward, menu-driven operation, HELP functions, prompts, and messages to facilitate simple and rapid programming while keeping the user fully informed of everything occurring at each step of a sequence. Programming capabilities should include capability to modify the electronics package's adjustable parameters, to test device diagnostics, and to store and retrieve telemetered data. It is desirable that when the implanted unit is interrogated, the present state of the adjustable parameters is displayed on the PC monitor so that the programmer may then conveniently change any or all of those parameters at the same time; and, if a particular parameter is selected for change, all permissible values for that parameter are displayed so that the programmer may select an appropriate desired value for entry into the pulse generator.

Other desirable features of appropriate software and related electronics would include the capability to store and retrieve historical data, including patient code, device serial number, number of hours of battery operation, number of hours of output, and number of magnetic activations (indicating patient intercession) for display on a screen with information showing date and time of the last one or more activations.

Diagnostics testing should be implemented to verify proper operation of the device, and to indicate the existence of problems such as with communication, the battery, or the lead/electrode impedance. A low battery reading, for example, would be indicative of imminent end of life of the battery and need for implantation of a new device. However, battery life should considerably exceed that of other implantable medical devices, such as cardiac pacemakers, because of the relatively less frequent need for activation of the pulse generator of the present invention. In any event, the nerve electrodes are capable of indefinite use absent indication of a problem with them observed on the diagnostics testing.

The device may utilize circadian or other programming as well, so that activation occurs automatically at normal mealtimes for this patient. This may be in addition to the provision for the manual, periodic between meal, and sensing-triggered activation as described above herein.

The pulse generator may also be activated manually by the patient by any of various means by appropriate implementation of the device. These techniques include the patient's use of an external magnet, or of an external RF signal generator, or tapping on the surface overlying the pulse generator, to activate the pulse generator and thereby cause the application of the desired modulating signal to the electrodes. Another form of treatment of may be implemented by programming the pulse generator to periodically deliver the vagal activity modulation productive of glycemic control at programmed intervals.

In some embodiments, the system may include one or more sensors that may provide for signals to initiate therapy signals to one or more electrodes. For example, a sensor may measure the amount of biological activity in the blood and initiate a signal to a nerve or organ if the amount exceeds or falls below a predetermined threshold.

As described herein methods are directed to modify biological activity selected from a group comprising blood glucose, heart rate, blood pressure, respiration, motion, CNS neuronal electrical signals, peripheral nerve signals and combinations thereof. In some example embodiments, a method of modifying the amount of biological activity comprises: applying an first intermittent (or continuous) electrical signal to a target nerve (or organ), with said first electrical signal selected to block or stimulate neural activity on the nerve and to restore neural activity on the nerve upon discontinuance of said signal, wherein the electrical signal is selected to modify the amount of biological activity being determined through a sensor.

In some embodiments, the method further comprises applying a second electrical signal treatment intermittently (or continuously) to a second target nerve or organ, wherein the second electrical signal has a frequency selected to block or stimulate activity on the target nerve or organ and to restore neural activity of the second target nerve (or organ) to restore activity of the target to baseline levels.

In another aspect of the disclosure, a system for treating a patient is provided. In some embodiments, the system comprises: at least one electrode operably connected to an implantable pulse generator, wherein one of the electrodes is adapted to be placed on a target nerve; an implantable pulse generator that comprises a power module and a programmable therapy delivery module, wherein the programmable therapy delivery module is configured to deliver at least one therapy program comprising an electrical signal treatment applied intermittently (or continuously) multiple times in a day and over multiple days to the target nerve, wherein the electrical signal has a frequency selected to alter activity on the target nerve and has an on time and an off time, wherein the off time is selected to allow at least a partial recovery of the activity of the target nerve; and an external component comprising a communication system and a programmable storage and communication module, wherein programmable storage and communication module is configured to store the at least one therapy program and to communicate the at least one therapy program to the implantable pulse generator.

In some embodiments, the programmable therapy delivery module is configured to deliver a second therapy program comprising an electrical signal treatment applied intermittently multiple times in a day and over multiple days to a second target nerve or organ, wherein the electrical signal has a frequency selected to upregulate or down-regulate activity on the target nerve and has an on time and an off time, wherein the off time is selected to allow at least a partial recovery of the activity of the target nerve or organ. In other related embodiments, the communication module is configured to store the at least one therapy program and to communicate the at least one therapy program to the implantable pulse generator using a communication system selected from a group consisting of an antenna, blue tooth technology, radio frequency, WIFI, light, sound and combinations thereof such as blue tooth technology, radio frequency, WIFI, light or sound.

### Signal Frequency and Timing

In some embodiments, a downregulating signal has a frequency of at least 200 Hz and up to 5000 Hz. In other embodiments, the signal is applied at a frequency of about 500 to 5000 Hz. Applicant has determined a most preferred blocking signal has a frequency of 3,000 Hz to 5,000 Hz or greater applied by two or more bi-polar electrodes. Such a signal has a preferred pulse width of 100 micro-seconds (associated with a frequency of 5,000 Hz). It is believed this frequency and pulse width best avoid neural recovery from blocking and avoid repolarization of the nerve by avoiding periods of no signal in the pulse cycle. A short “off time in the pulse cycle (e.g., between cycles or within a cycle) could be acceptable as long as it is short enough to avoid nerve repolarization. The waveform may be a square or sinusoidal waveform or other shape. The higher frequencies of 5,000 Hz or more have been found, in porcine studies, to result in more consistent neural conduction block. Preferably, the signal is bi-polar, bi-phasic delivered to two or more electrodes on a nerve.

In some embodiments, a signal amplitude of 0.01 to 20.0 mA is adequate for blocking. In other embodiments a signal amplitude of 0.01 to 10 mA is adequate for blocking. In still yet other embodiments a signal amplitude of 0.01 to 8 mA is adequate for blocking. Other amplitudes may suffice. Other signal attributes can be varied to reduce the likelihood of accommodation by the nerve or an organ. These include altering the power, waveform or pulse width.

Upregulating signals typically comprise signals of a frequency of less than 200 Hz, more preferably between 0.01 to 200 Hz, more preferably 10 to 50 Hz, more preferably 5 to 20 Hz, more preferably 5 to 10 Hz, more preferably 1 to 5 Hz, preferably 0.1 to 2 Hz, most preferably 1 Hz. Such a signal has a preferred pulse width of 0.1-10 microseconds. In some embodiments, a signal amplitude of 0.1 to 12 mA is adequate for stimulating. Other amplitudes may suffice. Other signal attributes can be varied to reduce the likelihood of accommodation by the nerve or an organ. These include altering the power, waveform or pulse width.

Selection of a signal that upregulates and/or downregulates neural activity and/or allows for recovery of neural activity can involve selecting signal type and timing of the application of the signal. For example, with an electrode conduction block, the block parameters (signal type and timing) can be altered by the pulse generator and can be coordinated with the stimulating signals. The precise signal to achieve blocking may vary from patient to patient and nerve site. The precise parameters can be individually tuned to achieve neural transmission blocking at the blocking site.

In some embodiments, the signal has a duty cycle including an ON time during which the signal is applied to the nerve followed by an OFF time during which the signal is not applied to the nerve. For example, the on time and off times may be adjusted to allow for partial recovery of the nerve. In some cases, the downregulating and
upregulating signals can be coordinated so that the upregulating signals are applied when down regulating signals are not being applied such as when the upregulating signals are applied at specific times or due to sensed events. In some embodiments, a sensed event indicates that an upregulating signal is applied and a down regulating signal is not applied for a time period relating to the sensed event, e.g. blood glucose exceeding a certain threshold. In preferred embodiments, the signal is continuously being applied.

Numerous medical conditions can be treated through the neuromodulation of two or more neuronal targets. Various neuromodulation systems, including those described herein, can be used to achieve the desired neuromodulation. Table 1 below provides just some examples of at least first and second neuronal targets that can be neuromodulated (stimulated (Stim) or conduction blocked (Block)) for treatment of a medical condition.

TABLE 1

| First Neuronal Target | Second Neuronal Target | Medical Condition |
|---|---|---|
| Cardio Splenic | Cervical Vagus Nerve-Stim | Heart Failure |
| Nerve-Block | | |
| Vagus Nerve-Stim | Greater and/or Lesser | Inflammation |
| | Splanchic Nerve-Block | |
| Baroreceptors-Stim | Renal Nerves-Block | Hypertension |
| Vagus Nerve-Block | Greater and/or Lesser | Pancreatitis |
| | Splanchic Nerve-Stim | |
| Vagus Nerve-Block | Sympathetic Nerves of | Refractory Asthma |
| | Smooth Muscles | |
| | of the Lungs | |
| | Innervation-Stim | |
| Sacral Nerves-Stim | Lumbar Sympathetic | Urinary |
| | Nerves-Block | Incontinence |
| Sacral Nerves-Stim | Lumbar Sympathetic | Erectile |
| | Nerves-Block | Dysfunction |
| Vago-Vagal Reflex | Satiety Afferents-Stim* | Obesity |
| (A Delta Fibers | | |
| of the Sub- | | |
| Diaphragmatic Vagus | | |
| Nerve)-Block | | |
| Subthalamic Nucleus | Cortex-Block | Parkinson's |
| (STN)-Stim | | Disease |
| Reward Centers-Stim | Vagus Nerve-Block | Obesity |
| Brodmann Area | Vagus Nerve-Stim | Refractory |
| 25-Stim or Block | | Depression |
| Vagus Nerve-Stim | Cortex-Block | Refractory Epilepsy |
| Occipital Nerve-Stim | Trigeminal Nerve- | Cluster Headaches |
| | Block (or Ablation) | |
| Afferents of Peripheral | Efferents of Peripheral | Obesity and |
| Nerve-Stim | Nerve-Block | Other Various |
| | | Medical Conditions |
| Afferents of Peripheral | Efferents of Peripheral | Various Medical |
| Nerve-Block | Nerve-Stim | Conditions |
| Celiac Nerve | Hepatic Nerve (or Anterior | Type II Diabetes |
| (or Posterior | Vagus Nerve above the | |
| Vagus Nerve above the | Branching Point of the | |
| Branching Point of the | Hepatic Nerve) | |
| Celiac Nerve)-Stim | | |

*Third Neuronal Target-Gall Bladder (Stim) for Bile Production

It should be appreciated that where in other embodiments a second target for electrical stimulation would be smooth muscle. Targets would include, but not limited to, the stomach, duodenum, small intestine, large intestine, bowel and bladder for the treatment of a medical condition. For example the antrum of the stomach and/or the duodenum could be stimulated to increase gastric emptying and decrease absorption of mono-and poly-saccharides for the treatment of type 2 diabetes. Antrum and/or duodenal stimulation could be combined with sub-diaphragmatic vagus nerve stimulation to further increased gastric emptying. Also, blocking of fibers innervating the liver would decrease insulin resistance and be used with antrum and/or duodenal stimulation.

Stimulation of the intestinal tract could be used to treat diseases, such as, but not limited to, Crohn's disease, which can produce chronic intestinal obstruction. Stimulation of nerves innervating the enteric nervous system as well as intestinal stimulation may be used to treat chronic intestinal obstruction.

Neuromodulation in combination of smooth muscle could also be used to treat diseases involved with voiding. Stimulation of the bladder in combination of blocking the pudendal nerve, which innervates the external ureteral sphincter, would induce voiding in patients with spinal cord injuries. Stimulation of the colon in combination of blocking inferior rectal nerves innervating the external anal sphincter could be used to increase elimination of feces in patients with spinal cord injuries. Stimulation of the pudendal nerve could be used for the treatment of urinary incontinence and stimulation of inferior rectal nerves could be used to treat fecal incontinence.

The results of the study suggest that a device which uses high frequency conduction block of the anterior sub-diaphragmatic vagal nerve trunk above the level of the hepatic branch and low frequency stimulation of the posterior sub-diaphragmatic vagal nerve trunk above the level of the celiac branch may be an effective method of glycemic control and a therapy for type II diabetes.

It will be appreciated that aspects of the various embodiments disclosed herein may be combined in any way to provide numerous additional embodiments. These embodiments will not be described individually for the sake of brevity.

A specific example illustrating the neuromodulation of two neuronal targets, e.g., the celiac nerve and hepatic nerve, for treatment of a medical condition, e.g. Type II Diabetes, through use of a neuromodulation system is provided below.

### EXAMPLE: TYPE II DIABETES—RESULTS OF RAT MODEL STUDY

The incidence of type II diabetes (which accounts for 90% of the cases of diabetes, World Health Organization (WHO)) is on the rise with significant consequences. In 2012 1.5 million deaths were attributed to diabetes and 2.2 million deaths were related to high blood glucose (WHO). In the US diabetes is the 6^{th} leading cause of death. People living with type II diabetes for years can develop neuropathic pain, nerve degeneration, blindness, and amputation. Treatments are limited and there is a large unmet need for novel therapies.

Type II diabetes takes a large toll on the body due to the toxic effect of sustained high blood glucose concentration. One such organ, the pancreas, plays a key role in stabilizing high blood glucose concentration. With prolonged high blood sugar levels, islet cells of the pancreas have a diminished ability to produce insulin which further decreases the body's ability to cope with high glucose. Finding a method to stabilize blood glucose levels for type II diabetics would stop this harmful cycle.

With the above in mind, a study was performed to determine the possible effectiveness of controlling glucose levels through stimulation of the posterior vagus nerve at the level of the celiac, e.g. a first neuronal target, in combination with a block of the hepatic nerve, e.g. a second neuronal target. More specifically, a differential modulation procedure was tested by using an IV glucose challenge (injection of a high concentration of glucose into the circulatory system) following a hepatic nerve vagotomy, or high frequency alternating current conduction block, with concurrent stimulation of the posterior vagus nerve at the level of the celiac branch in a rat model.

### Methods

Rats (250-300 grams) were divided into a control (n=5) and 2 experimental groups (n=5 for stimulation/vagotomy and 6 for stimulation/5000 Hz). In all groups, rats were fasted for at least 18 hours similar to other studies investigating changes in blood glucose concentrations (Lee and Miller, 1985). A sham surgery was performed in the control group. First, the rats were anesthetized with an intramuscular injection of a combination of ketamine, xylazine and acepromazine. Reflex testes were periodically preformed to determine that the rat was anesthetized and if the rat was lightly conscious an injection of ketamine was given. Next, the abdominal cavity was opened and the liver retracted. The hepatic nerve branch of the anterior vagal trunk (which will be referred to as the “hepatic branch”) and the celiac nerve branch of the posterior vagus nerve (which will be referred to as the “celiac branch”) were isolated and, using gentle dissection, were separated from the esophagus for the sham procedure. In the experimental groups, a similar surgical approach was performed as in the sham procedure except that the celiac branch was positioned on a bipolar platinum hook electrode for electrical stimulation and combined with vagotomy of the hepatic branch in the first experimental group and an electrode was positioned underneath the hepatic nerve to achieve conduction block combined with stimulation of the celiac branch in the second experimental group.

Blood samples were taken by wrapping a warm cloth around the tail of the rat (to stimulate blood flow) followed by cutting the end of the tail of the rat. The tail was then “milked” by squeezing from the end connecting the tail to the body of the rat to the cut end to ensure fresh systemic blood was sampled. An AlphaTrak (Abbott Laboratories, North Chicago, IL, USA) blood glucose monitor was used to measure blood glucose concentrations (mg/dl) from the milked rat tail.

A blood glucose measurement was taken at the start of the experiment and considered baseline. In experimental group 1, the hepatic branch was cut and the celiac branch was stimulated immediately following baseline sampling at a rate of 1 Hz. A piece of synthetic interstitial fluid (SIF) soaked gauze was placed between the hooks of the stimulation electrode to insure that no desiccation of the nerve occurred during the course of the experiment. A square wave with a pulse width of 4 millisecond was generated by a grass s44 stimulator (Grass Medical Instruments, Quincy, MA, USA) which drove a stimulus isolation unit (Model A360, World Precision Instruments, Sarasota, FL, USA). The pulse amplitude was 9 mA. In experimental group 1, the stimulation protocol was given for 1 hour with periodic sampling of blood glucose concentration. For the sham group, no stimulation was delivered to the celiac branch and the hepatic branch was not cut, but blood samples were taken with the same time course as the experimental group.

In experimental group 2, the celiac branch was stimulated with the same procedure as experimental group 1. However, instead of a hepatic nerve vagotomy, a 5000 Hz alternating current signal was applied to the hepatic branch with a bipolar platinum iridium hook electrode (the “blocking electrode”). The device delivering the 5000 Hz signal was a rechargeable neural regulator (RNR) similar to one used in the EnteroMedics' Recharge Clinical Study. A piece of SIF-soaked gauze was placed between the hooks of the blocking electrode to insure that no desiccation of the nerve occurred during the course of the experiment. The soaked gauze also decreased the impedances between the bipolar blocking electrodes to a level that was in the safety range of the RNR. The current amplitude was 12 mA with a typical impedance of 1000 ohms (measured at 1000 Hz at 3 mA). Voltages were measured with a portable fluke (Everett, WA, USA) oscilloscope across the blocking electrodes and were typically around 7 volts. This was smaller than the anticipated 12 volts which would be predicted by the impedance test. Previous experiments have determined that the differences between the anticipated and measured voltages are due to the differences in frequency and current amplitude between the impedance test and the blocking signal. Following the baseline blood sample, stimulation of the celiac branch and delivery of 5000 Hz to the hepatic nerve was concurrently initiated. A blood sample was then taken at 5 and 15 minutes following the start of the experiment.

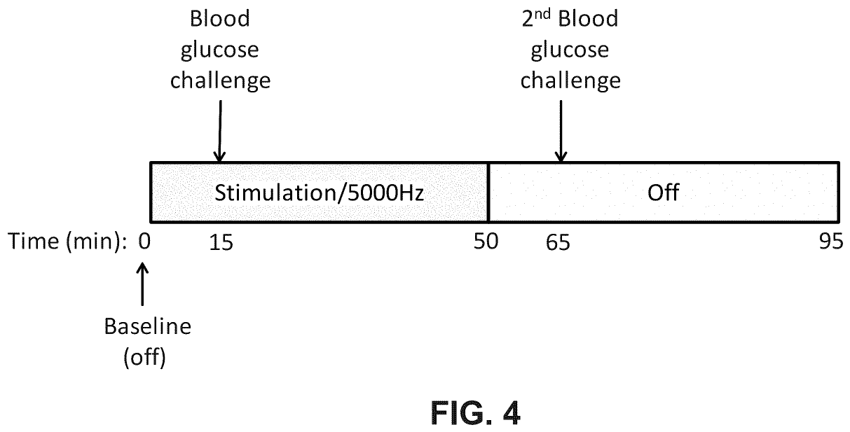

One hour following the initiation of experimental group 1 conditions (and sham) and 15 min following initiation of experimental group 2 conditions, a blood glucose challenge was performed. The blood glucose challenge consisted of an IV injection into the tail vein of a 0.5 g/kg dose of glucose made up in 0.9% saline with a 20% weight/volume concentration. Blood glucose was then sampled for 30 minutes following injection. Stimulation/vagotomy (experimental group 1) or stimulation/5000 Hz (experimental group 2) was continuously delivered during the glucose challenge and 30 minutes following. In 4 out of 6 rats in experimental group 2, a second glucose challenge was administered 15 minutes following the termination of the stimulation/5000 Hz procedure (FIG. 4).

Statistics consisted of a 2 tailed student's t-test assuming unequal variance performed with Microsoft Excel software (Redmond, WA, USA). All data are presented as mean±SEM. Percent change in glucose concentration was calculated using the following equation:

$$\% Change = ( ( Blood glucose concentration at time x - Baseline blood glucose concentration ) / ( Baseline blood glucose concentraton ) ) ⋆ 100$$

Area under the curve following the glucose challenge (% change in glucose concentration*time=area units) was calculated by assuming linearity between data points. The area between the line connecting two subsequent data points and the x-axis was calculated as one segment. The total number of segments following the glucose challenge was then summated.

### Results

### Experimental Group 1

### Effect of Hepatic Vagotomy and Celiac Branch Stimulation before Glucose Challenge

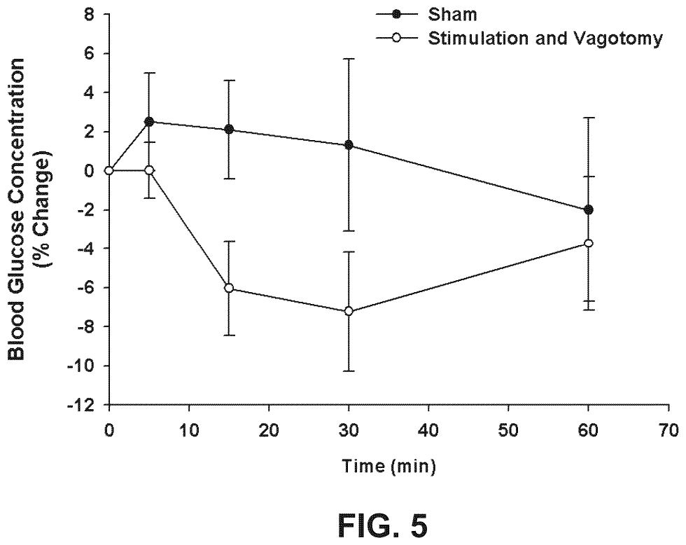

Referring now to FIG. 5, where blood glucose concentrations for the sham group remained relatively constant for the hour before the glucose challenge. At 5 min following stimulation/vagotomy in experimental group 1 there was no apparent change in glucose concentration. However, starting at 15 min there was a slight decrease in blood glucose concentration which lasted for the 60 min prior to the glucose tolerance test. As stated in the methods above, the rats were fasted and had a low initial blood glucose concentration (typically around 170 mg/dL) which may have caused a floor effect. Therefore, a glucose challenge was administered to test if the stimulation/vagotomy combination would give the rat the ability to tolerate a bolus injection of glucose into its circulatory system.

### Glucose Tolerance Test

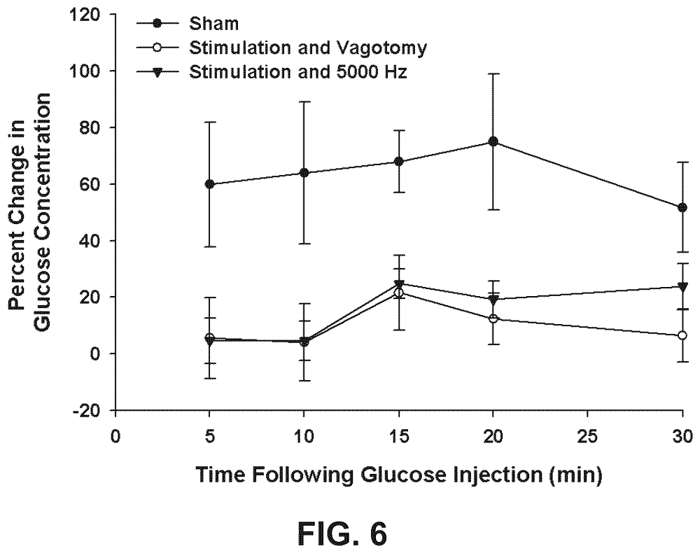

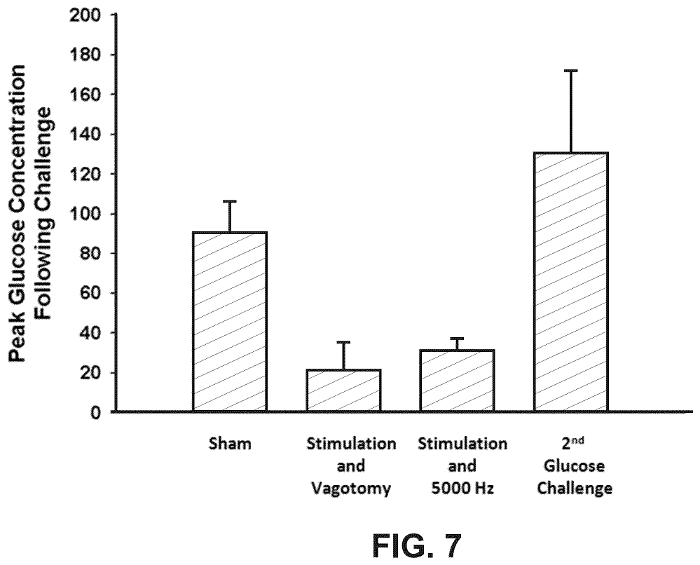

Refrring to FIG. 6, where five minutes following the glucose injection the sham group experienced, on average, a large increase in blood glucose concentration (60±22%) was shown. The time of peak glucose concentration was variable between sham rats. Referring now to FIG. 7, where the average in peak glucose concentration in sham was an increase of 90±16%. The glucose concentration remained high for the 30 minutes following the IV glucose injection with an apparent decrease at 30 minutes.

In the experimental group 1, 5 minutes following the glucose injection there was only a 5±14% increase in glucose concentration, on average (see FIG. 6). The glucose concentration remained relatively low for the 30 minutes following the glucose challenge with an average peak (21±13% increase) at 15 minutes post injection. As with the sham group, the time of the peak in glucose concentration was variable between rats. The average peak (21±14% increase) in experimental group 1 was significantly lower than sham (see FIG. 7, p=0.01).

The area under the curve of the % change in glucose concentration versus time following the glucose challenge was calculated as a measure of the total effect of the glucose tolerance test. This demonstrated a noticeable difference between the two groups (1704±553 area units for sham vs 202±322 area units for stimulation/vagotomy). Thus, not only was there a difference in the peak glucose concentrations between groups, but the total ability for experimental group 1 to tolerate a glucose challenge over time was strikingly greater than sham.

### Experimental Group 2

Experimental group 2 was performed using a 5,000 Hz alternating current applied to the hepatic nerve with concurrent stimulation of the celiac branch. This delivery significantly improved the ability to cope with a glucose challenge

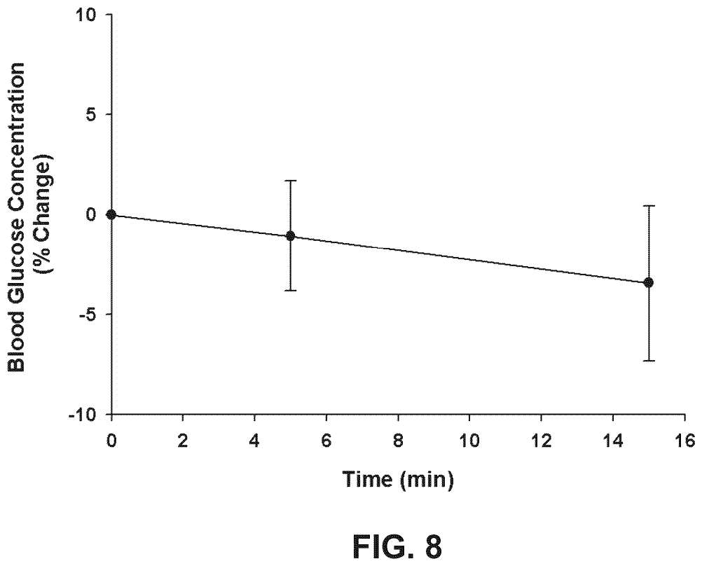

Referring now to FIG. 8, where a 5000 Hz alternating current signal will reversibly block conduction through sub-diaphragmatic vagus nerve in rat. Applying a 5000 Hz alternating current signal to the hepatic nerve while stimulating the celiac branch will have the same effect as cutting the hepatic nerve (with celiac branch stimulation) was tested. Similar to experimental group 1 there was a minor decrease in blood glucose concentration prior to the glucose challenge (see FIG. 8).

Following the glucose challenge blood glucose concentrations remained similar to experimental group 1 and noticeably lower than sham (see FIG. 6). The time of the peak in blood glucose concentration following the challenge varied in experimental group 2 similar to experimental group 1 and sham. The average peak was a 31±6% change from baseline which was significantly lower than sham (p=0.005, see FIG. 7). The ability of the rat to cope with the glucose challenge in experimental group 2 was also evident in its area under the curve value compared to sham (1704±553 area units for sham vs 418±140 area units for stimulation/5000 Hz).

### Reversibility of the Stimulation/5000 Hz Procedure

The large advantage of using 5000 Hz to block conduction through the hepatic nerve versus a complete hepatic vagotomy is that 5000 Hz conduction block is reversible allowing for normal communication from the brain to the liver (and vice versa) to occur at controlled times. To test if delivering 5000 Hz to the hepatic nerve along with stimulation of the celiac branch is reversible, the signals were turned off 30 minutes following the glucose challenge and 15 minutes later a subsequent glucose challenge was performed, referring to the timeline of the procedure in FIG. 4, where 4 out of the 6 rats in experimental group 2 were subject to this electrical delivery protocol.

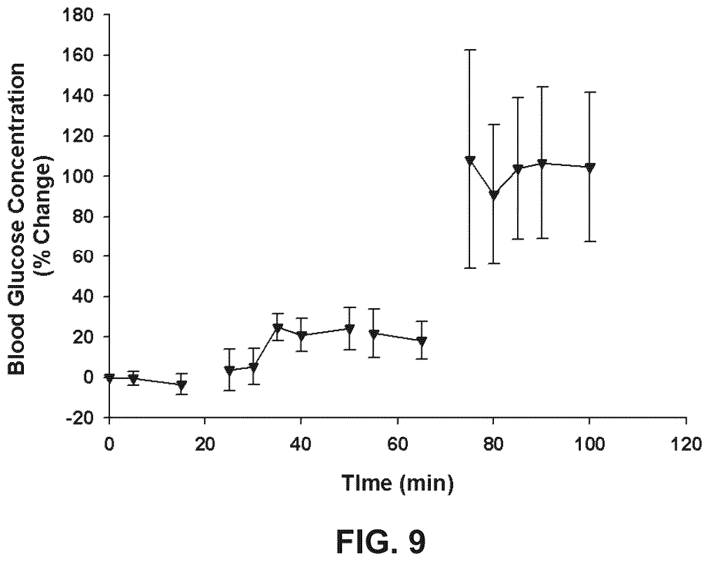

Referring now to FIG. 9, where 15 minutes post stimulation/5000 Hz block, blood glucose concentrations remained relatively stable. Following the second blood glucose challenge at time 15 minutes post stim/block (with all electrical signals turned off), there was a large increase in blood glucose concentration. As with all other glucose challenges in this study, the time of the peak blood glucose concentration varied between rats. The average peak was a 130±42% increase which was not significantly different from sham, but had a slight apparent increase. Area under the curve was also not significantly different than sham (1704±553 area units for sham vs 2566±915 area units for the 2^{nd} glucose challenge). It should be noted that 1 out of the 6 rats in experimental group 2 had a large increase in blood glucose concentration (similar to sham) following the first glucose challenge. However, it should be appreciated that the particular subject animal at issue was likely due to the fact that to prevent the nerves from desiccation, SIF soaked gauze was placed between the hooks of the stimulation and blocking electrodes. SIF is conductive and it was likely that the blocking electrode and/or the stimulation electrode had shorted out during this experiment. Thus, this anomaly data was considered an outlier and not included in the data set.

The data above demonstrates that by blocking neuronal information traveling to and from the liver while stimulating the celiac nerve (at the branching point from the posterior vagus nerve) significantly lowers blood glucose concentration following a glucose tolerance test compared to sham. Further, by using 5000 Hz to block neuronal information down the hepatic nerve (versus a vagotomy), and stimulation of the celiac branch, makes this procedure reversible. The stimulation can always be turned off and it is well established that high frequency conduction block is reversible. This was demonstrated by the rat's biological system behaving in a similar manner as the sham group (large increase in blood glucose following a glucose challenge) after cessation of the stimulation/5000 Hz procedure.

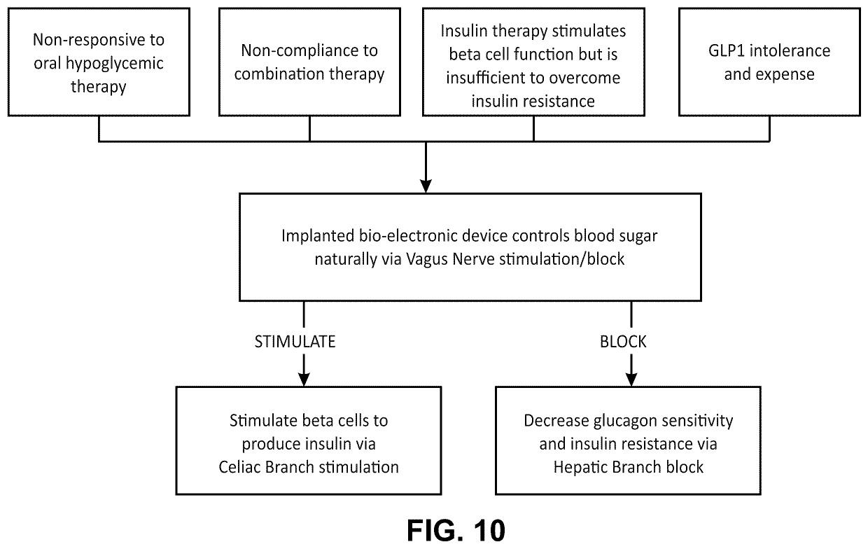

Referring now to FIGS. 10, where a flow chart depicts the needs for alternative treatment therapies for the stimulation and block for the treatment of hyperglycemia in Type-2 Diabetes. As shown the various needs or problems to be solved include, for example: Type 2 Diabetes patients do not respond to oral hypoglycemic therapy effectively after 3-5 years, particularly Sulfonylureas (301), Non-compliant Type 2 diabetic patents that are on combination therapy (302), diet and exercise suffer from uncontrolled blood sugars and HbA1c greater than 8, Type 2 diabetic patients that are on insulin therapy have beta cell function to produce endogenous insulin but it is not enough to overcome insulin resistance, including many patients also have non-alcoholic liver disease and problems with hypoglycemia (303) and GLP1 agonist are expensive and have tolerance issues such as nausea and GI issues. Therefore the solution is a novel implantable bio-electronic device to control blood sugar and their body's natural insulin secretion (305). This is performed using a stimulation (320) and/or block (330).

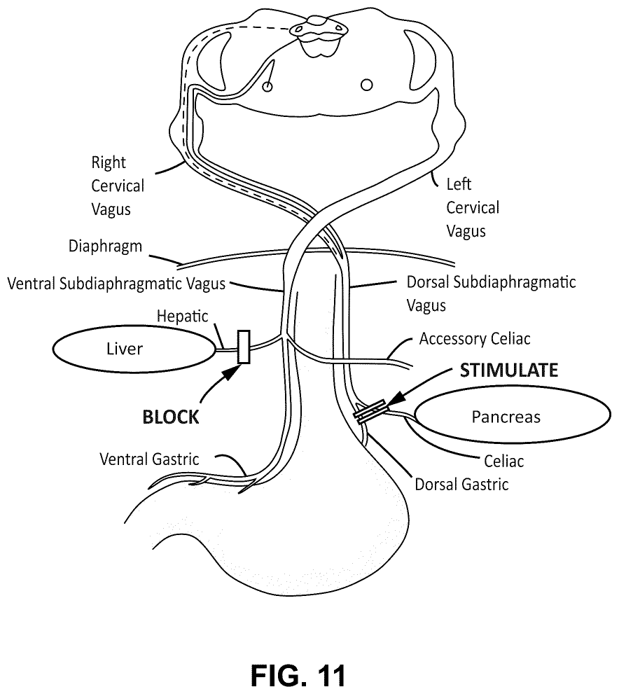

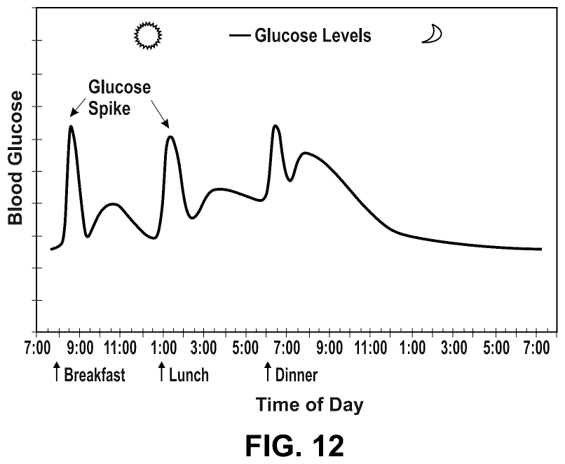

Referring now to FIG. 11, where the block and stimulation of the liver and pancreas respectively provide a dual modulation option which provides (1) pacing stimulation of the pancreas to release insulin; (2) Electrical block of hepatic branch to decrease glucose release from liver and (3) localized, reversible and customizable therapies. Referring now to FIG. 12 which provides a graphical illustration of Type 2 diabetics experience harmful “glucose spikes” throughout the day which leads to vascular and microvascular co-morbidities.

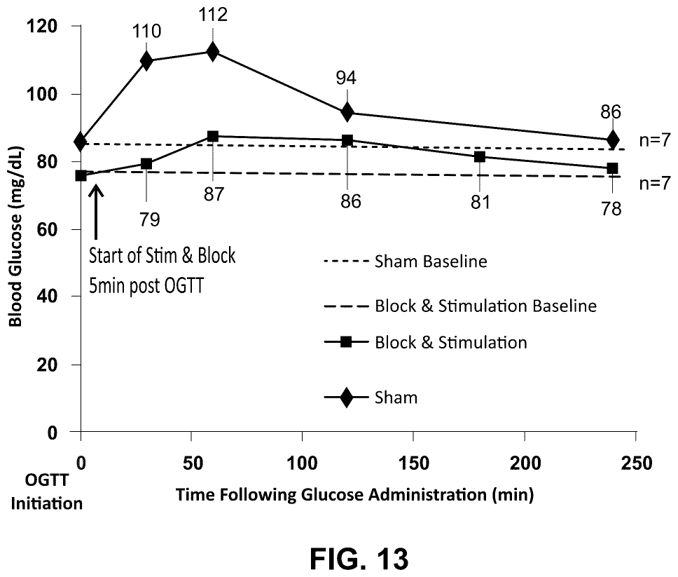

Referring now to FIG. 13 which shows Type II diabetes study described herein, of the blood glucose concentration (mg/dL) over time (min) with stimulation of the celiac branch and concurrent delivery of 5000 Hz to the hepatic nerve 5 minutes after Oral Glucose Tolerance Test (OGTT). The specific blood glucose values are shown in the graph in brackets “[ ]”. The data indicates that after 5 minutes following the start of the OGTT demonstrating the feassbility of this technology not running continugously be utilizing a continuous glucose monitoring system. This is also indicative of algorithm based technologies to provide predicative block and/or stimulation therpaies based on blood glucose data received in the closed loop system.

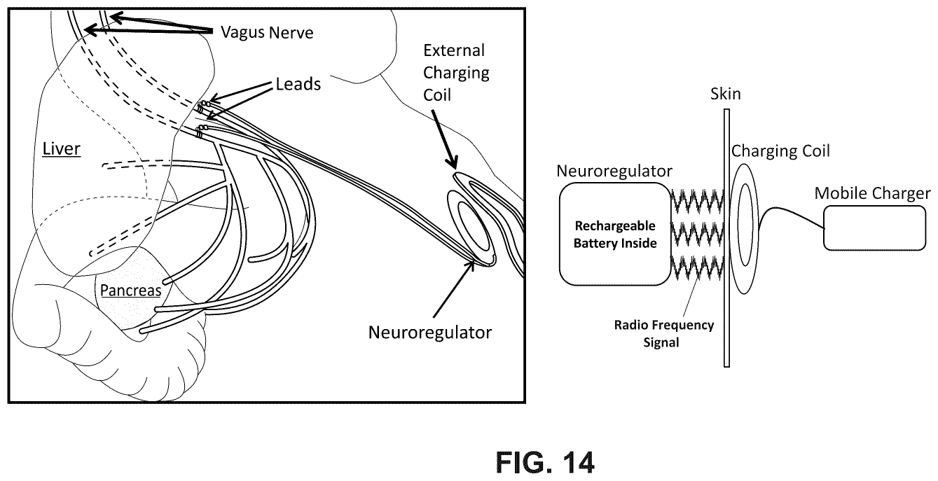

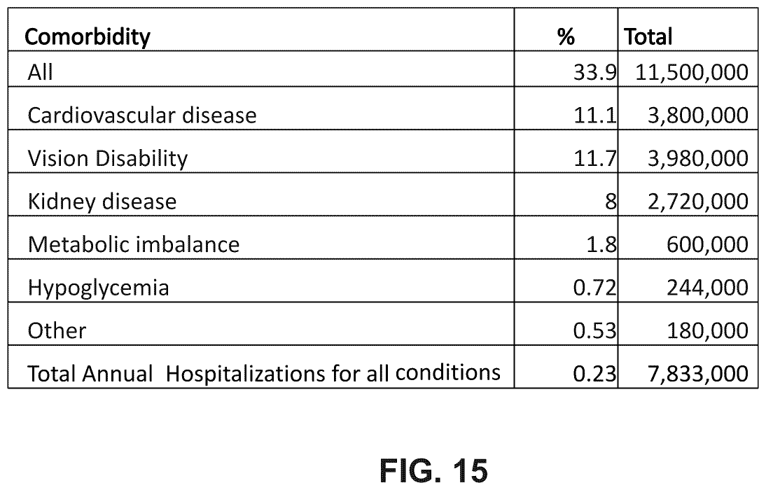

Referring now to FIG. 14, which shows anatomical placement of a system (410) for modifying biological activity. The system comprises a charging coil (416) and neuroregulator system (402) which is shown with a battery (430). As shown, the charging coil (416) is connected to a mobile charger (412). In at least this example embodiment, the system provides the patient with a radio frequency signal carrying energy to allow for periodic charging without the need for a transdermal charging port. Referring now to FIG. 15, where a table of diabetic co-morbitities are shown. The percentages in FIG. 15 are shown are derived from the CDC 2020 National Diabetes Statistics Report.

While the present invention has been described above primarily with reference to the accompanying drawings, it will be appreciated that the invention is not limited to the illustrated embodiments; rather, these embodiments are intended to disclose the invention to those skilled in this art. In the drawings, like numbers refer to like elements throughout.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention.

Well-known functions or constructions may not be described in detail for brevity and/or clarity. As used herein the expression “and/or” includes any and all combinations of one or more of the associated listed items.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes” and/or “including” when used in this specification, specify the presence of stated features, operations, elements, and/or components, but do not preclude the presence or addition of one or more other features, operations, elements, components, and/or groups thereof.

Herein, the terms “attached”, “coupled”, “connected”, “interconnected”, “contacting”, “mounted” and the like can mean either direct or indirect attachment or contact between elements, unless stated otherwise.

Herein describes non-limiting aspects of the invention through the below numbered clauses.

1. A system for regulating at least two neuronal targets:
a first neuroregulator that applies a stimulation or a conduction block to a first neuronal target; and
a second neuroregulator that applies a stimulation or a conduction block to a second neuronal target that is different from the first neuronal target, wherein the stimulation or conduction block of the first neuronal target works in cooperation with the stimulation or conduction block of the second neuronal target to treat a medical condition.
   2. The system of clause 1, wherein the first neuronal target and the second neuronal target each comprise one or more sympathetic nerves.
   3. The system of any one of clauses 1-2, wherein the first neuronal target and the second neuronal target each comprise one or more parasympathetic nerves.
   4. The system of any one of clauses 1-3, wherein the first neuronal target comprises one or more sympathetic nerves and wherein the second neuronal target comprises one or more parasympathetic nerves.
   5. The system of any one of clauses 1-4, wherein the stimulation or conduction block applied to the first neuronal target is of a different duration than that stimulation or conduction block applied to the second neuronal target.
   6. The system of any one of clauses 1-5, wherein the first neuroregulator applies the stimulation to the first neuronal target and the second neuroregulator applies the stimulation to the second neuronal target.
   7. The system of any one of clauses 1-6, wherein the first neuroregulator applies the conduction block to the first neuronal target and wherein the second neuroregulator applies the conduction block to the second neuronal target.
   8. The system of any one of clauses 1-7, wherein the first neuroregulator applies the conduction block to the first neuronal target and wherein the second neuroregulator applies the stimulator to the second neuronal target.
   9. The system of clause 8, wherein the first neuronal target comprises a hepatic nerve and the second neuronal target comprises the celiac branch of the posterior vagus nerve.
   10. The system of any one of clauses 1-9, wherein the first neuroregulator is an electrical neuroregulator.
   11. The system of clause 10, wherein the second neuroregulator is an electrical neuroregulator.
   12. The system of clause 10, wherein the second neuroregulator is a non-electrical neuroregulator.
   13. A method for treating a medical condition, comprising:
neuromodulating, with a stimulation or a conduction block, a first neuronal target; and
neuromodulating, with a stimulation or a conduction block, a second neuronal target, wherein the second neuronal target is different from the first neuronal target, and wherein the neuromodulation of the first neuronal target works in cooperation with the neuromodulation of the second neuronal target to treat a medical condition.
   14. The method of clause 13, wherein the first neuronal target and the second neuronal target each comprise one or more sympathetic nerves.
   15. The method of any one of clauses 13-14, wherein the first neuronal target and the second neuronal target each comprise one or more parasympathetic nerves.
   16. The method of any one of clauses 13-15, wherein the first neuronal target comprises one or more sympathetic nerves and wherein the second neuronal target comprises one or more parasympathetic nerves.
   17. The method of any one of clauses 13-16, wherein neuromodulating the first neuronal target comprises neuromodulating with the stimulation and wherein neuromodulating the second neuronal target comprises neuromodulating with the stimulation.
   18. The method of any one of clauses 13-17, wherein neuromodulating the first neuronal target comprises neuromodulating with the stimulation and wherein neuromodulating the second neuronal target comprises neuromodulating with the conduction block.
   19. The method of any one of clauses 13-18, wherein neuromodulating the first neuronal target comprises neuromodulating with the conduction block and wherein neuromodulating the second neuronal target comprises neuromodulating with the conduction block.
   20. A system for regulating at least two neuronal targets:
a first implantable electrical neuroregulator that applies a stimulation or a conduction block to a first neuronal target; and
a second implantable electrical neuroregulator that applies a stimulation or a conduction block to a second neuronal target that is different from the first neuronal target, wherein the stimulation or conduction block of the first neuronal target works in cooperation with the stimulation or conduction block of the second neuronal target to treat a medical condition.
   21. A method of sensing biological activity to trigger neuromodulation the method comprising the steps of:
obtaining a change in biological activity from a sensor;
initiating the neuromodulator to initiate at least one signal to a target nerve, organ or tissue, thereby altering the biological activity to a predetermined level, where the signal is stopped.
   22. The method of clause 21, wherein the method is a closed-loop system.
   23. The method of clause 21, wherein the method is an open-looped system.
   24. The method of any one of clauses 21-23, wherein the signal is from at least two electrodes.
   25. The method of any one of clauses 21-24, wherein the at least two electrodes are initiated at the same time.
   26. The method of any one of clauses 21-24, wherein the at least two electrodes are initiated at different times.
   27. The method of any one of clauses 21-24, wherein the at least two electrodes are initiated, wherein one electrical signal initiates a block and the other electrical signal initiates a stimulation.
   28. The method of any one of clauses 21-24, wherein the at least two electrodes are initiated, wherein one electrical signal initiates a block and the other electrical signal initiates a block.
   29. The method of any one of clauses 21-24, wherein the at least two electrodes are initiated, wherein one electrical signal initiates a stimulation and the other electrical signal initiates a stimulation.
   30. The method of any one of clauses 21-29, wherein the sensor detects a change in biological activity selected from a group comprising blood glucose, heart rate, blood pressure, respiration, motion, CNS neuronal electrical signals, peripheral nerve signals and combinations thereof.

Although exemplary embodiments of this invention have been described, those skilled in the art will readily appreciate that many modifications are possible in the exemplary embodiments and methods without materially departing from the novel teachings and advantages of this invention. Accordingly, all such modifications are intended to be included within the scope of this invention as defined in the claims. The invention is defined by the following claims, with equivalents of the claims to be included therein.

1. A system for regulating at least two neuronal targets:
a first neuroregulator that applies a stimulation or a conduction block to a first neuronal target; and
a second neuroregulator that applies a stimulation or a conduction block to a second neuronal target that is different from the first neuronal target, wherein the stimulation or conduction block of the first neuronal target works in cooperation with the stimulation or conduction block of the second neuronal target to treat a medical condition.

2. The system of claim 1, wherein the first neuronal target and the second neuronal target each comprise one or more sympathetic nerves.

3. The system of claim 1, wherein the first neuronal target and the second neuronal target each comprise one or more parasympathetic nerves.

4. The system of claim 1, wherein the first neuronal target comprises one or more sympathetic nerves and wherein the second neuronal target comprises one or more parasympathetic nerves.

5. The system of claim 1, wherein the stimulation or conduction block applied to the first neuronal target is of a different duration than that stimulation or conduction block applied to the second neuronal target.

6. The system of claim 1, wherein the first neuroregulator applies the stimulation to the first neuronal target and the second neuroregulator applies the stimulation to the second neuronal target.

7. The system of claim 1, wherein the first neuroregulator applies the conduction block to the first neuronal target and wherein the second neuroregulator applies the conduction block to the second neuronal target.

8. The system of claim 1, wherein the first neuroregulator applies the conduction block to the first neuronal target and wherein the second neuroregulator applies the stimulator to the second neuronal target.

9. The system of claim 8, wherein the first neuronal target comprises a hepatic nerve and the second neuronal target comprises the celiac branch of the posterior vagus nerve.

10. The system of claim 1, wherein the first neuroregulator is an electrical neuroregulator.

11. The system of claim 10, wherein the second neuroregulator is an electrical neuroregulator.

12. The system of claim 10, wherein the second neuroregulator is a non-electrical neuroregulator.

13. A method for treating a medical condition, comprising:
neuromodulating, with a stimulation or a conduction block, a first neuronal target; and
neuromodulating, with a stimulation or a conduction block, a second neuronal target, wherein the second neuronal target is different from the first neuronal target, and wherein the neuromodulation of the first neuronal target works in cooperation with the neuromodulation of the second neuronal target to treat a medical condition.

14. The method of claim 13, wherein the first neuronal target and the second neuronal target each comprise one or more sympathetic nerves.

15. The method of claim 13, wherein the first neuronal target and the second neuronal target each comprise one or more parasympathetic nerves.

16. The method of claim 13, wherein the first neuronal target comprises one or more sympathetic nerves and wherein the second neuronal target comprises one or more parasympathetic nerves.

17. The method of claim 13, wherein neuromodulating the first neuronal target comprises neuromodulating with the stimulation and wherein neuromodulating the second neuronal target comprises neuromodulating with the stimulation.

18. The method of claim 13, wherein neuromodulating the first neuronal target comprises neuromodulating with the stimulation and wherein neuromodulating the second neuronal target comprises neuromodulating with the conduction block.

19. The method of claim 13, wherein neuromodulating the first neuronal target comprises neuromodulating with the conduction block and wherein neuromodulating the second neuronal target comprises neuromodulating with the conduction block.

20. A system for regulating at least two neuronal targets:
a first implantable electrical neuroregulator that applies a stimulation or a conduction block to a first neuronal target; and
a second implantable electrical neuroregulator that applies a stimulation or a conduction block to a second neuronal target that is different from the first neuronal target, wherein the stimulation or conduction block of the first neuronal target works in cooperation with the stimulation or conduction block of the second neuronal target to treat a medical condition.

21. A method of sensing biological activity to trigger neuromodulation the method comprising the steps of:
obtaining a change in biological activity from a sensor;
initiating the neuromodulator to initiate at least one signal to a target nerve, organ or tissue, thereby altering the biological activity to a predetermined level, where the signal is stopped.

22-24. (canceled)

25. The method of claim 21, wherein the signal is from at least two electrodes and wherein the at least two electrodes are initiated at the same time.

26. (canceled)

27. The method of claim 21, wherein the at least two electrodes are initiated in a signally protocol selected from a group consisting of one electrical signal initiates a block and the other electrical signal initiates a stimulation, one electrical signal initiates a block and the other electrical signal initiates a block and one electrical signal initiates a stimulation and the other electrical signal initiates a stimulation.

28-29. (canceled)

30. The method of claim 21, wherein the sensor detects a change in biological activity selected from a group comprising blood glucose, heart rate, blood pressure, respiration, motion, CNS neuronal electrical signals, peripheral nerve signals and combinations thereof.
